(19) United States
(12) Patent Application Publication
(10) Pub. No.: US 2019/0175554 A1
(43) Pub. Date: Jun. 13, 2019

(54) METHOD FOR TREATING PRURITUS AND/OR ITCH

(71) Applicant: Neurim Pharmaceuticals (1991) Ltd., Tel Aviv (IL)

(72) Inventor: Moshe LAUDON, Kfar Saba (IL)

(73) Assignee: Neurim Pharmaceuticals (1991) Ltd., Tel Aviv (IL)

(21) Appl. No.: 16/324,707

(22) PCT Filed: Jun. 30, 2017
(86) PCT No.: PCT/IB2017/053989

§ 371 Date: Feb. 11, 2019

Related U.S. Application Data
(60) Provisional application No. 62/378,435, filed on Aug. 23, 2016.

(51) Int. Cl. A61K 31/4045; A61K 45/06; A61P 17/04; A61K 9/06; A61K 47/44; A61K 31/404; A61K 47/46; A61K 47/26; A61K 47/22; A61K 47/38; A61K 47/36; A61K 47/24

(52) U.S. Cl. CPC A61K 31/4045; A61K 45/06; A61P 17/04; A61K 9/06; A61K 47/44; A61K 47/24; A61K 47/46; A61K 47/26; A61K 47/22; A61K 47/38; A61K 47/36; A61K 31/404

(57) ABSTRACT

The invention relates to a medicament for treating pruritus and itch in a patient suffering from dermatological and non-dermatological conditions leading to such symptoms, which comprises at least one compound selected from a pyrone-indole derivative, in an effective amount, and optionally one or more other therapeutically active agents.

### CROSS-REFERENCE TO RELATED APPLICATION

This application is a 37 U.S.C. 37 National Phase Entry from PCT/IB2017/053989, filed Jun. 30, 2017, which claims the benefit under 35 U.S.C. § 119(e) of the filing date of provisional patent application Ser. No. 62/378,435 filed Aug. 23, 2016, the disclosures of which are incorporated herein by reference in their entirety.

### FIELD OF THE INVENTION

The present disclosure relates generally to treating pruritus and/or itch, to the use of pyrone-indole compounds in the manufacture of a medicament useful for treating pruritus and/or itch, and to a medicament for use in alleviating itch or pruritus in a patient suffering from dermatological and non-dermatological conditions leading to such symptoms.

### BACKGROUND OF THE INVENTION

Itch is an uncomfortable sensation on the skin that causes a desire to scratch (Bautista, Wilson et al. 2014). Pruritus, defined as a sensation driving the urge to scratch, may be acute (<6 weeks) or chronic (>6 weeks) (Lavery, Stull et al. 2016). This symptom can significantly impair the quality of life and sleep of affected patients. Moreover the cumulative effect of such disruptions may influence mortality. The dialysis outcomes and practice patterns (DOPPS) study showed a 17% increased mortality risk among pruritic hemodialysis patients, which was attributed in part to decreased quality of sleep. Pruritus is severe itching of the skin, as a symptom of various ailments. Itch can be classified by etiology (Chuquilin, Alghalith et al. 2016). Pruriceptive itch originates from the activation of primary afferent nerve terminals (e.g., insect bites). Neurogenic itch is caused by central nervous system injury or activation without the activation of sensory nerve terminals (e.g., renal disease and kidney failure). Psychogenic itch results from a pure central psychic processing disorder in the absence of skin pathology or underlying medical disease.

A myriad of both dermatological and non-dermatological conditions may cause itch and/or pruritus. In clinical practice, patients with chronic pruritus from any cause commonly complain of nocturnal involvement. Nocturnal pruritus is common in patients with chronic itch and negatively affects sleep quality. Lack of sleep can have both immediate and long-term effects that lead to medical, social and financial ramifications. Physicians must be aware of the numerous etiologies of itch and nocturnal pruritus and deliver treatment with a patient-centered approach. Aptly tailored management can help alleviate distress and reduce the complications that result from repetitive scratching.

Table 1 depicts some of the more common etiologies of pruritus.

| Category | Disease |
|---|---|
| Dermatological | Atopic dermatitis |
| | Psoriasis |
| | Chronic idiopathic urticaria |
| | Infestations (scabies, bed bugs, pediculosis, |
| | pinworms) |
| | Lichen planus |
| | Lichen simplex chronicus |
| | Prurigo nodularis |
| | Advanced age (senile) pruritus |
| | Brachioradial pruritus |
| Non-Dermatological | Liver disease |
| | Chronic kidney disease |
| | Hematopoietic disorders |
| | Neurological (e.g., brachioradial pruritus) |
| | Psychological (delusional ideations, depression, |
| | schizophrenia, stress) |
| | Substance abuse |
| | Advanced age (senile) pruritus |
| | Restless legs syndrome |

Many patients suffer from nocturnal pruritus, which can decrease quality of life and affect mortality in hemodialysis patients (Lavery, Stull et al. 2016). Nocturnal pruritus may occur in all sleep stages but is most prevalent in stages N1 and N2. Conditions with a high prevalence of nocturnal pruritus include advanced age (senile) pruritus, atopic dermatitis, prurigo nodularis, psoriasis, brachioradial pruritus and chronic idiopathic urticaria. Two major dermatological conditions that cause nocturnal pruritus are atopic dermatitis and psoriasis. Atopic dermatitis is a chronic condition with a prevalence of 7.2% in adults and 10.7% in children in the USA. Chronic pruritus is one of the most common presenting complaints in atopic dermatitis patients with a point prevalence ranging from 87%-100% (Lauffer and Ring 2016). Clinically, atopic dermatitis manifests as pruritic erythematous lesions with associated excoriations, lichenification and/or superimposed infection. Psoriasis is a chronic inflammatory immune-mediated skin disorder. Prevalence varies greatly by country with a prevalence of 0.91% in the USA and 8.5% in Norway. Activation of the immune system triggers cytokine cascades involved in the different psoriatic cutaneous manifestations. Chronic idiopathic urticaria (also called chronic spontaneous urticaria) is defined as itchy hives that last for at least 6 weeks, with or without angioedema, and that have no apparent external trigger. The condition generally has a prolonged duration of 1 to 5 years (persisting for >5 years in 11 to 14% of patients) and has a detrimental effect on patients' emotional and physical health-related quality of life. The impairment accompanying this disorder has been likened to that seen in patients with ischemic heart disease, with patients feeling a similar lack of energy, social isolation, and emotional upset as those with heart disease. (Maurer, Rosen et al. 2013).

Itch neurons (pruriceptors) are polymodal and respond to stimulus other than pruritogens, including heat and capsaicin (Chuquilin, Alghalith et al. 2016). Although pruriceptive neurons are a subset of nociceptive C-fiber neurons in DRG, recent progress indicates that there are separate labeled lines for itch and pain in the spinal cord. The selectivity theory of itch is the most largely accepted theory to explain why a stimulus is able to cause itch and not pain. Pruriceptors are a subset of nociceptors, and this theory postulates that itch occurs when these selective itch neurons are activated alone, while the sensation of pain dominates when itch and pain neurons are activated together. In this manner, inhibition of the itch pathway occurs via the nociceptive only neurons.

Numerous itch mediators (pruritogens) and receptors (pruriceptors) have been identified, of which the best understood are histamine, proteases, opioids, substance P, the Mas-related G protein-coupled receptor (Mrgpr) family, and calcitonin gene-related peptide (CGRP). Circadian rhythms may play a role in nocturnal pruritus. The hypothalamus-pituitary-adrenal axis is a complex system that releases corticosteroids through a continuous negative feedback mechanism.

H1-antihistamines are the current mainstay for initial treatment and are the only agents approved for use in patients with chronic idiopathic urticarial and nocturnal pruritus (Furue and Kadono 2015). However, a majority of patients do not have a response to H1-antihistamines, even when the drugs are administered at three to four times their approved doses. Treatment options for patients who do not have a response to H1-antihistamines include the use of H2-antihistamines, leukotriene-receptor antagonists, systemic glucocorticoids, cyclosporine, hydroxychloroquine, dapsone, methotrexate, sulfasalazine, and intravenous immune globulin. Mirtazapine, an atypical antidepressant that antagonizes noradrenergic (α1, α2), serotonergic (5-HT2, 5-HT3), and histaminergic (H1) receptors, has demonstrated efficacy in relieving nocturnal itch of varied etiology. None of these agents has yet received regulatory approval for the treatment of itch. In addition, the data supporting the use of these drugs are limited, and long-term use of some of the agents can be associated with substantial side effects.

Transient receptor potential vanilloid type 1 (TRPV1) is a non-selective cation channel widely expressed in skin tissues, and peripheral sensory nerve fibers. Activation of TRPV1 releases neuropeptides; the resulting neurogenic inflammation is believed to contribute to the development of pruritus. However, in a recent study topical administration of a TRPV1 antagonist failed to be of symptomatic benefit for histaminergic or non-histaminergic induced itch in humans (Gibson, Robertson et al. 2014).

Smooth muscle cells of cutaneous arterioles and arteriole-venule shunts (AVS) in the skin express sodium channel Nav1.7. Moreover, Nav1.7 is expressed by endothelial cells lining the arterioles and AVS and by sensory and sympathetic fibers innervating these vascular elements. (Rice, Albrecht et al. 2015). Recent studies discovered that the NaV1.7 sodium channel plays a key role in spinal cord nociceptive and pruriceptive synaptic transmission. These studies reveal that NaV1.7 is a target for itch management and an anti-NaV1.7 antibody has therapeutic potential for suppressing pain and itch. (Lee, Park et al. 2014).

There is a need in the field for an effective method and medicament for treating pruritus and/or itch. Surprisingly, the present inventors have found that certain pyrone-indole compounds have inhibitory properties at Nav1.7 and TRPV1 channels and may be useful for the treatment of pruritus and itch. Such compounds are described in U.S. Pat. Nos. 7,635,710, 8,242,163 and 8,569,355 and International Patent Specification Nos. WO2007/093880A2 and WO2007/093880A3. The entire contents of the above-mentioned patents and literature references are incorporated herein by reference.

### SUMMARY OF THE INVENTION

The present disclosure provides in one aspect, use of at least one pyrone-indole compound selected from compounds described in U.S. Pat. Nos. 7,635,710, 8,242,163 and 8,569,355 and International Patent Specification Nos. WO2007/093880A2 and WO2007/093880A3, the entire contents of these references are incorporated herein by reference, in an effective amount, in the manufacture of a medicament, for treating itch and/or pruritus, in a patient suffering from dermatological and non-dermatological conditions leading to such symptoms, wherein the medicament additionally comprises at least one pharmaceutically acceptable diluent, preservative, antioxidant, solubilizer, emulsifier, adjuvant or carrier.

In another aspect, the present disclosure provides a method for treating a subject suffering from itch and/or pruritus by administering an effective amount of at least one compound of this disclosure to the subject.

In still another aspect, the present disclosure provides a medicament for use in a patient suffering from dermatological and non-dermatological conditions leading to itch and/or pruritus. The medicament composition contains an effective amount of at least one compound of this disclosure and at least one additional therapeutic agent selected from UV protectants, hypnotics, sedatives, analgesics, minor tranquilizers, and anti-inflammatory drugs, in addition to at least one pharmaceutically acceptable diluent, preservative, antioxidant, solubilizer, emulsifiers adjuvant or carrier.

### BRIEF DESCRIPTION OF THE DRAWINGS

FIG. 1 shows a stimulus voltage pattern used for determining blocking effects on hNav1.x channels.

### DETAILED DESCRIPTION OF THE INVENTION

In one aspect, the present disclosure includes a method for treating a patient suffering from itch and/or pruritus by administering an effective amount of a pharmaceutical composition containing a compound having the formula (I):

Ar—B—Ar′ (I)

wherein:
—B— represents:

### X—Y—Z—

wherein:
X represents —(CH₂)ₙ— (wherein n is 0-6), in which the alkyl moiety is linear or branched,
Y represents oxygen, sulphur, >NH or is absent;
Z represents >C=0, or >0, or >COO or is absent;
wherein at least one of X, Y and Z must be present;
ring system Ar represents an indole nucleus:

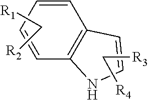

ring system Ar′ represents an alpha-, beta- or gamma-pyrone nucleus:

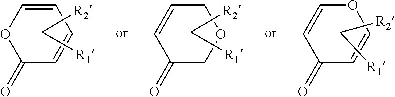

wherein each of the R₁₋₄ substitutes the ring systems Ar at any available position (including the N-position) and each of the R_{1′-2′} substitutes the ring system Ar′ at any available position and wherein each of R₁₋₄ and R_{1′-2′} independently represents hydrogen, oxygen, halo, halo-C₁₋₅ alkyl, aryl, acyl, a C₅₋₇ heterocyclic group containing 1-3 hetero atoms independently selected from nitrogen, oxygen and sulphur; a C₆₋₈ heteroaryl group containing 1-3 hetero atoms independently selected from nitrogen, oxygen and sulphur; C₁₋₅ alkyl, C₂₋₅ alkenyl, C₂₋₅ alkynyl, aryl-C₁₋₅ alkyl, aryl-C₂₋₅ alkenyl, aryl-C₂₋₅ alkynyl, hydroxy-C₁₋₅ alkyl, nitro, amino, cyano, cyanamido, guanidino, amidino, acylamido, C₁₋₅ alkylamine, C₁₋₅ alkylamido, hydroxy, thiol, acyloxy, azido, C₁₋₅ alkoxy, carboxy, carbonylamido or styryl; wherein said arylalkyl, arylalkenyl, arylalkynyl, or styryl group optionally can be ring-substituted by one to four substituents independently selected from the group consisting of hydrogen, halo, halo-C₁₋₅ alkyl, aryl, a C₅₋₇ heterocyclic group containing from 1-3 heteroatoms independently selected from nitrogen, oxygen and sulphur; a heteroaryl group containing from 1-3 hetero atoms independently selected from nitrogen, oxygen and sulphur; C₁₋₅ alkyl, C₂₋₅ alkenyl, C₂₋₅ alkynyl, aryl-C₂₋₅ alkenyl, aryl-C₂₋₅ alkynyl, hydroxy-C₁₋₅ alkyl, nitro, amino, cyano, cyanamido, guanidino, amidino, acylamido, hydroxy, thiol, acyloxy, azido, alkoxy, carboxy, carbonylamido, S-alkyl or alkylthiol; and either of R₃ or R₄ further can include or represent a bond to B;
wherein Ar can be bonded to B at any position on the Ar ring not substituted by R₁ and R₂ including the N-position, and Ar′ can be bonded to B at any carbon on the Ar′ ring not substituted by R_{1′} or R_{2′},
or a pharmaceutically acceptable salt, stereoisomer, or racemic mixture thereof.

As used herein, “aryl” represents phenyl or naphthyl.

Also as used herein, reference to “a” compound, salt, stereoisomer, or racemic mixture of formula (I) is intended to encompass “one or more” such compounds, salts or stereoisomers. Furthermore, reference to a “compound” of formula (I), as in the discussion below of pharmaceutical formulations, is also intended to include a salt, stereoisomer, or racemic mixture of the compound.

In a preferred embodiment, X is —(CH₂)ₙ—, wherein n is any of 0-6 and preferably any of 1-6, Y is >NH or >O and Z is >CO.

Without prejudice to the generality of the compounds of the present invention, in a preferred embodiment of the compounds defined by formula (I), X is —(CH₂)₂—, Y is >NH or >O, Z is >C═O, Ar is an indole containing a bond, R₃ to X at position 3 of the indole ring, R₁ is methoxy on position 5 of the indole ring, each of R₂ and R₄ is hydrogen, Ar′ is a gamma-pyrone bonded to Z at position 2 of the pyrone ring, R₁ is hydrogen or a hydroxy group at position 5 of the pyrone ring and R₂′ is hydrogen or a carboxy group at position 6 of the gamma pyrone ring; or a pharmaceutically acceptable salt, stereoisomer, or racemic mixture thereof. In a second preferred embodiment, Ar is as defined above and Ar′ is an alpha-pyrone ring bonded to Z at position 5 of the alpha-pyrone ring and R₁ and R_{2′} are hydrogens; or a pharmaceutically acceptable salt, stereoisomer, or racemic mixture thereof.

Suitable pharmaceutically acceptable salts of the compounds of formula (I) include salts which may, for example, be formed by mixing a solution of the compound with a solution of a pharmaceutically acceptable acid. Pharmaceutically acceptable acids include, but are not limited to hydrochloric acid, fumaric acid, maleic acid, succinic acid, acetic acid, citric acid, benzoic acid, tartaric acid, carbonic acid, phosphoric acid or sulfuric acid. Salts of amine groups may also comprise the quaternary ammonium salts in which the amino nitrogen atom carries an alkyl, alkenyl, alkynyl or aralkyl group. Where the compound carries an acidic group, for example a carboxylic acid group, the present invention also contemplates salts thereof, preferably non-toxic pharmaceutically acceptable salts thereof, such as the sodium, potassium and calcium salts thereof. Representative pharmaceutically acceptable salts include, yet are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

In some embodiments, the functional groups of the compounds of formula (I) useful in the invention can be modified to enhance the pharmacological utility of the compounds. Such modifications are well within the knowledge of a person of ordinary skill in the art and include, without limitation, esters, amides, ethers, N-oxides, and pro-drugs of the indicated compound of formula (I). Examples of modifications that can enhance the activity of the compounds of formula (I) include, for example, esterification such as the formation of C₁ to C₆ alkyl esters, preferably C₁ to C₄ alkyl esters, wherein the alkyl group is a straight or branched chain. Other acceptable esters include, for example, C₁ to C₇ cycloalkyl esters and arylalkyl esters such as benzyl esters. Such esters can be prepared from the compounds described herein using conventional methods well known in the art of organic chemistry.

It is understood that, in embodiments where the compounds of formula (I) useful in the invention have at least one chiral center, the compounds can exist as chemically distinct enantiomers. In addition, where a compound has two or more chiral centers, the compound can exist as diastereomers. All such isomers and mixtures thereof are encompassed within the scope of the indicated compounds of formula (I). Similarly, where the compounds possess a structural arrangement that permits the structure to exist as tautomers, such tautomers are encompassed within the scope of the indicated compound. Furthermore, in crystalline form, the compounds may exist as polymorphs; in the presence of a solvent, a compound may form a solvate, for example, with water or a common organic solvent. Such polymorphs, hydrates and other solvates also are encompassed within the scope of the invention as defined herein.

In one embodiment, the present disclosure includes a cream formulation for topical application comprising shea butter, coconut oil, and a therapeutically effective amount of a pyrone-indole derivative. The cream formulation may include N-[2-(1H-indol-3-yl)-ethyl]-comanilamide. The concentration of the N-[2-(1H-indol-3-yl)-ethyl]-comanilamide may be, e.g., 1-10%, 2-8%, 3-5%, or 3%.

A pharmaceutical composition useful in the methods of the invention can be administered to a subject by a variety of means depending, for example, on the type of pain to be treated, the compound to be included in the composition, and the history, risk factors and symptoms of the subject. The compounds of the invention may be administered by oral, parenteral (e.g., intramuscular, intraperitoneal, intravenous or subcutaneous injection, or implant), intrapulmonary (e.g., by inhalation), nasal, rectal, transbuccal, transdermal, or topical routes of administration. The compounds may also be administered by electrophoresis; topically in any acceptable form such as in drops, creams, gels or ointments; and by minipump or other implanted prolonged release device or formulation. “Prolonged release” refers to release of an active agent from a dosage form at a rate effective to achieve a therapeutic amount of the agent, or active metabolite thereof, locally or in the systemic blood circulation over a prolonged period of time. Release of the agent occurs over an extended period of hours, for example, over a period of at least 6 hours, over a period of at least 8 hours, over a period of at least 12 hours, or over a period of at least 24 hours. It is understood that different means of drug delivery can be combined in a method of the invention. As an example, intravenous administration on a first day can be combined with oral or topical dosing on a second and/or third day.

The term “subject” or “patient” as used herein, refers to a vertebrate, including but not limited to fish (such as commercially farmed fish, pet fish, etc.), amphibians (such as frogs, toads, pet amphibians, etc.), reptiles (such as snakes, lizards, turtles, pet reptiles, etc.), birds (such as chickens, turkeys, pet birds, etc.) and mammals (such as mice, rats, hamsters, rabbits, pigs, dogs, cats, horses, cows, sheep, goats, non-human primates, non-human mammals, pet non-human mammals, humans, etc.). In certain embodiments, the subject or patient is a mammal. In certain embodiments, the subject or patient is a mouse, a rat, a hamster, a rabbit, a pig, a dog, a cat, a horse, a cow, a sheep, a goat, a non-human primate, or a human (which may be included in embodiments of the invention individually or in any combination). In certain embodiments, the subject or patient is a human. In certain embodiments, the subject or patient is a non-human mammal.

Solid dosage forms for oral administration include capsules, tablets, pills, powders and granules. In such solid dosage forms, the active compound is admixed with at least one inert pharmaceutically acceptable carrier such as sucrose, lactose, or starch. Such dosage forms can also comprise, as is normal practice, additional substances other than inert diluents, e.g., lubricating agents such as magnesium stearate. Illustrative of the adjuvants which may be incorporated in tablets, capsules and the like are the following: a binder such as gum tragacanth, acacia, corn starch or gelatin; an excipient such as microcrystalline cellulose; a disintegrating agent such as corn starch, pregelatinized starch, alginic acid and the like; a lubricant such as magnesium stearate; a sweetening agent such as sucrose, lactose or saccharin; a flavoring agent such as peppermint, oil of wintergreen or cherry. In the case of capsules, tablets and pills, the dosage forms may also comprise buffering agents. When the dosage unit form is a capsule, it may contain, in addition to materials of the above type, a liquid carrier such as a fatty oil. Various other materials can be present as coatings or to otherwise modify the physical form of the dosage unit. Tablets and pills can additionally be prepared with enteric coatings and tablets may be coated with shellac, sugar or both.

Liquid dosage forms for oral administration include pharmaceutically acceptable emulsions, solutions, suspensions, syrups and elixirs containing inert diluents commonly used in the art, such as water. Besides such inert diluents, compositions can also include adjuvants, such as wetting agents, emulsifying and suspending agents, and sweetening, flavoring, and perfuming agents. A syrup or elixir may contain the active compound, sucrose as a sweetening agent, methyl and propyl parabens as preservatives, a dye and a flavoring such as cherry or orange flavor.

Preparations according to this invention for parenteral administration include sterile aqueous or non-aqueous solutions, suspensions, or emulsions. Sterile compositions for injection can be formulated according to conventional pharmaceutical practice by dissolving or suspending the active substance in a vehicle such as water for injection, a naturally occurring vegetable oil like sesame oil, coconut oil, peanut oil, cottonseed oil, etc., or a synthetic fatty vehicle like ethyl oleate or the like. Buffers, preservatives, antioxidants and the like may be incorporated as required. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions that can be dissolved in sterile water, or some other sterile injectable medium immediately before use.

The dosage of active agent in compositions of this disclosure can vary, provided that a therapeutic amount is administered. Such therapeutic amount generally is the minimum dose necessary to achieve the desired therapeutic effect, which can be, for example, that amount roughly necessary to reduce the itching to tolerable levels. Desirably the active agent is administered to a patient (human or animal) in need of such treatment in dosages that will provide optimal pharmaceutical efficacy. The selected dosage depends upon the nature and severity of the disease or disorder to be treated, desired therapeutic effect, the route of administration, and the duration of treatment. The dose will vary from patient to patient depending on the nature and severity of the disease, the patient's weight, special diets then being followed by the patient, concurrent medication, the bioavailability of the compound upon administration and other factors which those skilled in the art will recognize. Therapeutic doses are generally in the range of 0.1-1000 mg/day and can be, for example, in the range of 0.1-500 mg/day, 0.5-500 mg/day, 0.5-100 mg/day, 0.5-50 mg/day, 0.5-20 mg/day, 0.5-10 mg/day or 0.5-5 mg/day, with the actual amount to be administered determined by a physician taking into account the relevant circumstances including the severity of the itching, the age and weight of the patient, the patient's general physical condition, the cause of itching and the route of administration. In some embodiments, the therapeutically effective amount comprises a dosage of 0.10 mg, 0.15 mg, 0.20 mg, 0.25 mg, 0.5 mg, 0.75 mg, 1 mg, 2 mg, 2.5 mg, 3 mg, 4 mg, 5 mg, 6 mg, 7 mg, 8 mg, 9 mg, 10 mg, 15 mg, 20 mg, 25 mg, or 30 mg one or more times a day. As a non-limiting example, the compounds of the present invention may be administered by repeated dosing or continuous dosing over a period of at least three days, or for example, over three days, four days, five days, six days, seven days, eight days, nine days or ten days. As a further example, the compounds can be administered multiple times a day, such as twice per day, three times per day, four times per day or more.

In some embodiments, the dose of the compound is sufficient to reduce itch and/or pruritus by at least 30%. In other embodiments, itch and/or pruritus are reduced by at least 40%, 50%, 60%, 70%, 80%, 90%, or 100%.

A pharmaceutical composition useful in the invention includes the active compound (i.e. a compound of formula (I)) and further can include, if desired, an excipient such as a pharmaceutically acceptable carrier or a diluent, which is any carrier or diluent that has substantially no long term or permanent detrimental effect when administered to a subject. Such an excipient generally is mixed with active compound, or permitted to dilute or enclose the active compound. A carrier can be a solid, semi-solid, or liquid agent that acts as an excipient or vehicle for the active compound. Examples of pharmaceutically acceptable carriers and diluents include, without limitation, water, such as distilled or deionized water; saline; and other aqueous media. It is understood that the active ingredients can be soluble or can be delivered as a suspension in the desired carrier or diluent.

In certain embodiments, a pharmaceutical formulation for use according to the present disclosure is characterized by at least one of the following features:
i. it is adapted to be administered by oral, parenteral (e.g., intramuscular, intraperitoneal, intravenous or subcutaneous injection, or implant), intrapulmonary (e.g., by inhalation), nasal, rectal, transbuccal, transdermal, or topical routes of administration and can be formulated in dosage forms appropriate for each route of administration;
ii. it is in unit dosage form, each unit dosage in an effective amount;
iii. it is a prolonged release formulation.
iv. it is in a depot form which will release the compound slowly in the body, over a preselected time period;
v. it is an ointment, cream, foam or spray intended for topical use;
vi. it optionally includes at least one additional therapeutic agent selected from UV protectants, analgesics, minor tranquilizers, and anti-inflammatory drugs.

The present disclosure includes a composition containing at least one compound in an amount effective in treating itch and/or pruritus in a patient suffering from dermatological and non-dermatological conditions leading to such symptoms and at least one pharmaceutically acceptable diluent, preservative, antioxidant, solubilizer, emulsifiers adjuvant or carrier. More preferably, the composition is further characterized by at least one of the features (i), (ii), (iii), (iv) (v) and (vi), set forth above.

In the composition with which the present invention is concerned, the pharmaceutically acceptable diluents, preservatives, solubilizers, emulsifiers, adjuvants and carriers are those acceptable for pharmaceutical formulations. The pharmaceutical composition can include, if desired, one or more agents such as emulsifying agents, wetting agents, sweetening or flavoring agents, tonicity adjusters, preservatives, buffers or anti-oxidants. Tonicity adjustors useful in a pharmaceutical composition include salts such as sodium chloride, potassium chloride, mannitol or glycerin and other pharmaceutically acceptable tonicity adjustors. Preservatives useful in the pharmaceutical compositions of the invention include, without limitation, benzalkonium chloride, chlorobutanol, thimerosal, phenylmercuric acetate, and phenylmercuric nitrate. Various buffers and means for adjusting pH can be used to prepare a pharmaceutical composition, including, but not limited to, acetate buffers, citrate buffers, phosphate buffers and borate buffers. Similarly, anti-oxidants useful in the pharmaceutical compositions of the invention are well known in the art and include, for example, sodium metabisulfite, sodium thiosulfate, acetylcysteine, butylated hydroxyanisole and butylated hydroxytoluene. It is understood that these and other substances known in the art of pharmacology can be included in a pharmaceutical composition useful in the invention.

For oral administration, the medicament may be utilized as e.g. tablets, capsules, emulsions, solutions, syrups or suspensions. For parenteral administration, the medicament may be utilized in the form of ampoules, or otherwise as suspensions, solutions or emulsions in aqueous or oily vehicles or patch. For ectopic use (topical administration) the medicament may be utilized in the form of ointment, cream, gel, foam, solution, aerosol or spray. The medicament may be water-based (aqueous) or organic-based. The need for suspending, stabilizing and/or dispersing agents will of course take account of the fact of the solubility or otherwise of the active compounds, in the vehicles which are used in particular embodiments. The medicament may additionally contain e.g. physiologically compatible preservatives and antioxidants. The medicament may also be utilized as suppositories with conventional suppository bases such as cocoa butter or other glycerides.

As described above, the present disclosure includes co-administering the compounds described above at least one compound selected from such compounds described in U.S. Pat. Nos. 7,635,710, 8,242,163 and 8,569,355 and International Patent Specification No. WO2007093880A2 and WO2007093880A3, may be administered in conjunction with (i.e. simultaneously, separately or sequentially) other compounds which are known in the art to be useful for protecting the skin and for alleviating itch by reducing anxiety, improving sleep and reducing pain, including e.g., at least one additional therapeutic agent selected from hypnotics (benzodiazepines as well as non-benzodiazepines and melatonin agonists), sedatives, analgesics, minor tranquilizers, and anti-inflammatory drugs. Examples of such additional therapeutic agents are brotizolam, buspirone, diazepam, diphenhydramine, doxepin, fluvoxamine, clonidine, zolpidem, zopiclone, Melatonin, UV protectants (p-aminobenzoic acid, menthyl anthranilate, octyl methoxycinnamate, titanium dioxide, etc.) and pharmaceutically acceptable salts and combinations thereof.

The additional therapeutic agent may be e.g., an anti-inflammatory corticosteroid, such as dexamethasone or melatoninergic agents, at least one compound selected from melatonin, other melatonergic agents, melatonin agonists and/or antagonists, may be administered in conjunction with the use of physical methods such as with light therapy or electrical stimulation, e.g. scheduling bright light administration, ordinary-intensity light exposure, or exposure to dim-light or darkness.

It is understood that the instant disclosure encompasses compounds which inhibit, inter alia, channels involved in itch response. It is further understood that the instant disclosure encompasses compounds which are either selective or non-selective for such channels. In some embodiments, the compounds of the invention are highly selective for a channel involved in itch response, and are characterized as having a potency of less than 900 nM, 800 nM, 700 nM, 600 nM, 500 nM, 400 nM, 300 nM, 200 nM, 100 nM, 50 nM, 10 nM or 1 nM.

The present invention also provides a method of screening for effective agents that reduce itching, comprising identifying a compound believed to have having anti-itch activity, contacting the compound with a receptor involved in the itch response, and determining whether the compound has anti-itch activity.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled. All patents and publications cited herein are incorporated by reference.

The invention will be illustrated by the following Examples.

### EXAMPLES

### Example 1

### Synthesis of N-[2-(1H-indol-3-yl)-ethyl]-comanilamide

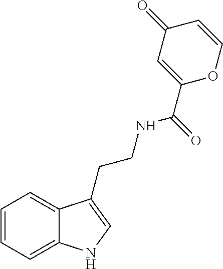 [SMILES: O=C(NCCc1c[nH]c2ccccc12)c1cc(=O)cco1]

Reaction Scheme for the Synthesis of N-[2-(1H-indol-3-yl)-ethyl]-comanilamide

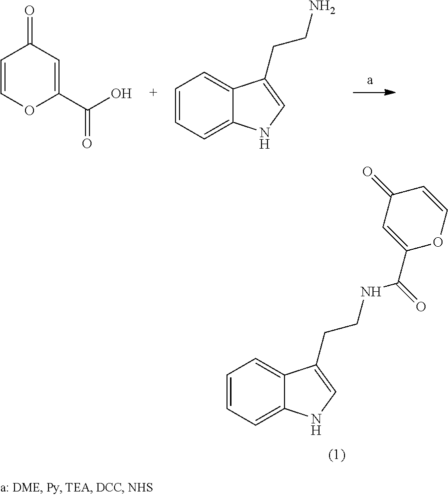 [SMILES: O=C(O)c1cc(=O)cco1 ; NCCc1c[nH]c2ccccc12 ; O=C(NCCc1c[nH]c2ccccc12)c1cc(=O)cco1]

General Procedure for the Synthesis of N-[2-(1H-indol-3-yl)-ethyl]-comanilamide

In a four necked round bottom flask (100 mL), tryptamine (0.85 g, 5.3 mmol)) was dissolved in DME (40 mL) and Py (0.9 mL) was added. The mixture was stirred 30 minutes at room temperature then comanic acid (0.68 g, 4.8 mmol) was added, followed by NHS (0.61 g, 5.3 mmol), DCC (1.1 g, 5.3 mmol), TEA (0.7 mL, 56.1 mmol); the mixture was stirred overnight, then checked by LC-MS. The brown suspension was filtered on a gooch, washing the solid with water (100 mL). The aqueous phase was extracted with CH₂Cl₂ (3×100 mL), washed with brine (2×10 mL), dried over MgSO₄, filtered and evaporated under reduced pressure. The obtained sticky solid was suspended and stirred in MTBE (20 mL) for 30 minutes. The solid was filtered and washed with CH₂Cl₂ to give 0.48 g (4.8 mmol, yield 36%) of product (1) as a light brown solid (LC-MS assay >95%).

Experimental data for N-[2-(1H-indol-3-yl)-ethyl]-comanilamide

MS: m/z=283 [M+H]+

TLC: (Cy/EtOAc 1:9) Rf=0.3

FTIR (cm-1): 3330, 2925, 2851, 2353, 2116, 1240, 937, 741.

1H NMR (DMSO): 2.91 (m, 2H); 3.55 (m, 3H); 6.42 (m, 1H); 6.79 (m, 1H); 6.99 (m, 1H); 7.07 (m, 1H); 7.19 (m, 1H); 7.34 (m, 1H); 7.58 (m, 1H); 8.21 (m, 1H); 9.05 (m, 1H); 10.83 (bs, 1H).

### Example 2

### Synthesis of N-[2-(5-methoxy-indol-3-yl)-ethyl]-comanilamide

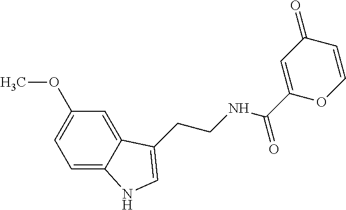 [SMILES: COc1ccc2[nH]cc(CCNC(=O)c3cc(=O)cco3)c2c1]

Reaction Scheme for the Synthesis of N-[2-(5-methoxy-indol-3-yl)-ethyl]-comanilamide

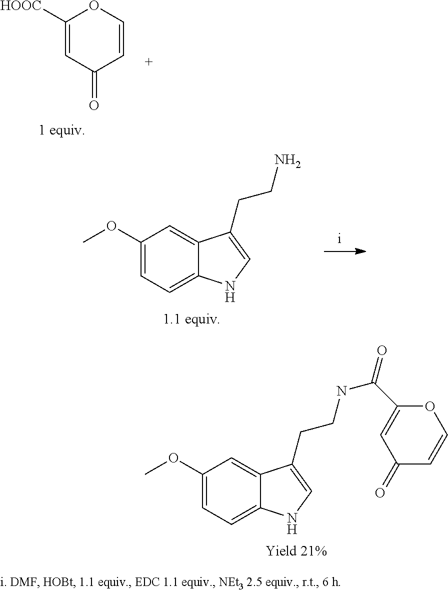 [SMILES: O=C(O)c1cc(=O)cco1 ; COc1ccc2[nH]cc(CCN)c2c1 ; COc1ccc2[nH]cc(CCNC(=O)c3cc(=O)cco3)c2c1]

General Procedure for the Synthesis of N-[2-(5-methoxy-indol-3-yl)-ethyl]-comanilamide

Under an argon atmosphere, a 100 ml three-necked flask round-bottom flask was charged with comanic acid (500 mg, 1 equiv.) and 5-methoxytryptamine (760 mg, 1.1 equiv.), dissolved in DMF (25 ml), and brought to 0° C. by means of an ice-bath. HOBt (1-hydroxybenxotriazole monohydrate, 530 mg, 1.1 equiv.), EDC (1-(3-dimethylaminopropyl)-3-ethylcarbodiimide hydrochloride, 750 mg, 1.1 equiv.) and triethylamine (1.25 ml, 2.5 equiv.) were then added under magnetic stirring. The mixture was stirred for an additional 15 minutes at 0° C. and subsequently allowed to react for 6 h at room temperature. The reaction course was followed by HPLC-MS. Water (50 ml) was then added and the mixture was extracted with dichloromethane (3×50 ml). The combined organic phases were dried over Na₂SO₄ and the solvent was removed by rotary evaporation. The crude was then chromatographed over a silica gel column by eluting with dichloromethane/methanol 95/5. The product was recovered as a bright yellow solid (235 mg, yield 21%).

Experimental data for N-[2-(5-methoxy-indol-3-yl)-ethyl]-comanilamide MS (ESI POS): 313 (M+H), 330 (M+H2O), 335 (M+Na), 376 (M+Na+CH3CN)

HPLC assay: 98%

1H NMR (DMSO-d6, 400 MHz) δ 2.88-2.92 (m, 2H, CH2CH2NH), 3.48-3.53 (m, 2H, CH2CH2NH), 3.75 (s, 3H, OCH3), 6.42 (dd, J1=2.3 Hz, J2=5.9 Hz, 1H, CH═CH), 6.71 (dd, J1=2.1 Hz, J2=8.8 Hz, 1H, aromatic H), 6.78 (d, J=2.3 Hz, 1H, aromatic H), 7.04 (d, J=2.3 Hz, 1H, CH), 7.13 (d, J=2.1 Hz, 1H, aromatic H), 7.22 (d, J=8.8 Hz, 1H, aromatic H), 8.21 (d, J=5.9 Hz, 1H, CH═CH—CO), 9.04 (br t, J=5.8 Hz, 1H, CH2CH2NH), 10.65 (br s, 1H, NH).

### Example 3

### Synthesis of 2-methyl-4-oxo-4H-pyran-3-yl [2-(5-methoxy-1H-indol-3-yl)ethyl]carbamate

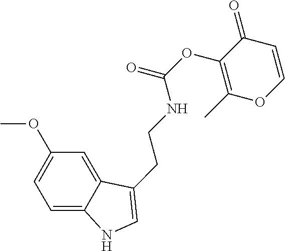 [SMILES: COc1ccc2[nH]cc(CCNC(=O)Oc3c(C)occc3=O)c2c1]

Reaction Scheme for the Synthesis of 2-methyl-4-oxo-4H-pyran-3-yl [2-(5-methoxy-1H-indol-3-yl)ethyl]carbamate

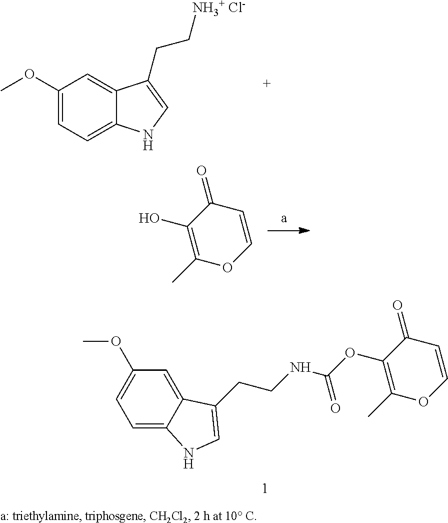

1:2-methyl-4-oxo-4H-pyran-3-yl[2-(5-methoxy-1H-indol-3-yl)ethyl]carbamate

To a suspension of 5-methoxytryptamine hydrochloride (1 g, 0.0044 mol) in CH₂Cl₂ (7 ml) triethylamine (1.8 ml, 0.0132 mol) was added and the mixture stirred for 30′ at room temperature. After the addition of maltol (0.5 g, 0.0044 mol) the reaction mixture was cooled in an ice-water bath then triphosgene (0.4 g, 0.0015 mol) in CH₂Cl₂ (1 ml) was added, keeping the temperature below 10° C., and stirred for 2 h at the same temperature. The mixture was evaporated under reduced pressure to give an oily residue that was purified by flash chromatography (EtOAc-Cy, 80:20 to ETOAc 100) giving 500 mg of pale-yellow solid. This solid was purified by a second flash chromatography (VersaPak C18, Spherical, 23×53 mm, H₂O-MeOH, 6:4) yielding 220 mg of white foam that was washed with MTBE to give 150 mg of 1 as a white solid.

Rf: 0.11 (EtOAc-Cy, 8:2); 0.47 (reversed phase; MeOH—H₂O, 6:4)

HPLC: tr=5.5 Assay >95%

MS (ESI+) m/z=343 [M+H]+

FTIR (cm-1): 3855; 3751; 3328; 2938; 2270; 1735; 1656; 1578; 1526; 1485; 1438; 1364; 1236; 1183; 1072; 1034; 923; 834; 798; 637; 566; 432.

1H-NMR (CDCl3): 2.25 (s, 3H), 3.01 (t, 2H), 3.57 (q, 2H), 3.86 (s, 3H), 5.29 (t, 1H), 6.39 (d, 1H), 6.85-6.88 (m, 1H), 7.05 (d, 1H), 7.13 (d, 1H), 7.26 (d, 1H), 7.65 (d, 1H), 8.05 (bs, 1H).

### Example 4

Formalin injected in the nape of the neck was previously reported to elicit significant hindlimb scratching in rats and mice, and formalin as well as pruritogens elicited enhanced scratching in mice lacking a population of inhibitory spinal interneurons, suggesting that formalin-evoked nocifensive behavior may reflect itch rather than, or in addition to, pain (Akiyama, Carstens et al. 2010). The anti-pruritus activity of pyrone-indole compounds were assessed using this model.

Method. The noxious stimulus is an injection of dilute formalin under the skin of the dorsal surface of the right hindpaw. The response is the amount of time the animals spend licking the injected paw 5-10 minutes (early phase), or 20-30 minutes (late phase) after the injection of formalin. Mice (N=8 per group) were injected intraperitoneally with test compound (100 mg/kg), or morphine (4 mg/kg), or vehicle. Thirty minutes later, formalin (20 μl 2.5% in water) was injected by the subplantar route into the right hindpaw. Hindpaw licking time was recorded in consecutive 5 minute-intervals during the first 10 minutes (early phase), and from 15 to 35 minutes (late phase) after formalin injection.

Results. The compound N-[2-(1H-indol-3-yl)-ethyl]-comanilamide resulted in inhibition of the licking response by 57% in the early and 90% in the late phase, both statistically different from vehicle response. The compound N-[2-(1H-indol-3-yl)-ethyl]-comanilamide resulted in inhibition of the licking response by 38% in the early and 62% in the late phase, both statistically different from vehicle response. The compound 2-methyl-4-oxo-4H-pyran-3-yl [2-(5-methoxy-1H-indol-3-yl)ethyl]carbamate resulted in inhibition of the licking response by 54% in the early and 86% in the late phase, both statistically different from vehicle response.

Conclusions. These results show beneficial effects of N-[2-(1H-indol-3-yl)-ethyl]-comanilamide, N-[2-(5-methoxy-indol-3-yl)-ethyl]-comanilamide and 2-methyl-4-oxo-4H-pyran-3-yl [2-(5-methoxy-1H-indol-3-yl)ethyl]carbamate on itch induced by formalin injection in the mouse. N-[2-(1H-indol-3-yl)-ethyl]-comanilamide has potential anti-itching effects in pruritus.

### Example 5

Effects of N-[2-(1H-indol-3-yl)-ethyl]-comanilamide, N-[2-(5-methoxy-indol-3-yl)-ethyl]-comanilamide and 2-methyl-4-oxo-4H-pyran-3-yl [2-(5-methoxy-1H-indol-3-yl)ethyl]carbamate on Nav1.x Channels Expressed in Mammalian Cells

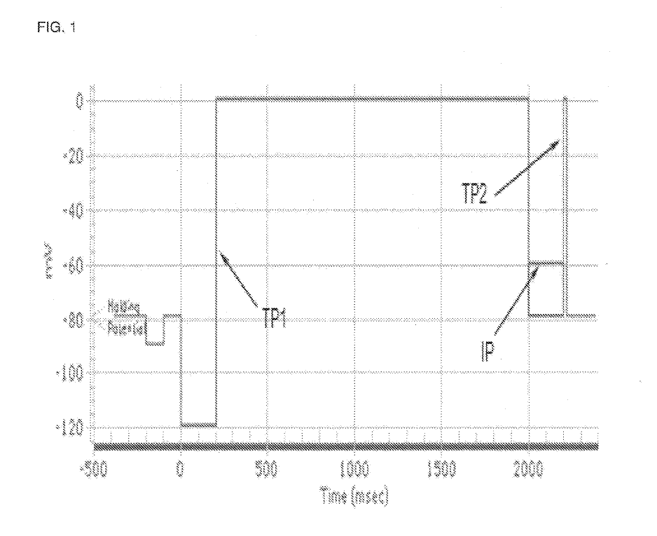

Method. A block of hNav1.x channels was measured using the stimulus voltage pattern shown in FIG. 1; voltage potentials are indicated in Table 2, below. The pulse pattern repeated twice: before and 5 minutes after N-[2-(1H-indol-3-yl)-ethyl]-comanilamide, N-[2-(5-methoxy-indol-3-yl)-ethyl]-comanilamide and 2-methyl-4-oxo-4H-pyran-3-yl[2-(5-methoxy-1H-indol-3-yl)ethyl]carbamate addition and peak current amplitudes at 3 test pulses was measured (ITP1 and ITP2).

Results. N-[2-(1H-indol-3-yl)-ethyl]-comanilamide, N-[2-(5-methoxy-indol-3-yl)-ethyl]-comanilamide and 2-methyl-4-oxo-4H-pyran-3-yl [2-(5-methoxy-1H-indol-3-yl)ethyl]carbamate blocking effects on hNav1.x channels (Table 3) showing preferential inhibition of Nav1.7 channels by N-[2-(1H-indol-3-yl)-ethyl]-comanilamide.

Conclusions. These results show inhibition of Nav1.7 channels that mediate itch response using the compounds of this disclosure.

TABLE 2
Voltage-protocol parameters for hNav1.x channels

| | | | | | 50-ms |
|---|---|---|---|---|---|
| | | 200-ms | 1800-ms | 200-ms | Test |
| | Holding | Pre-Pulse | Test | Inter | Pulse 2 |
| | Potential | Potential | Pulse 1 | Pulse | Potential |
| Channel | (mV) | (mV) | (mV) | (mV) | (mV) |
| Nav1.1−1.7 | −80 | −120 | 0 | −60 | 0 |
| Nav1.8/beta3 | −80 | −120 | 20 | −60 | 20 |

TABLE 3

| | | IC50, mM | | | | | | | |
|---|---|---|---|---|---|---|---|---|---|
| Test Article | Type of | | | | | | | | Nav1.8/ |
| ID | the block | Nav1.1 | Nav1.2 | Nav1.3 | Nav1.4 | Nav1.5 | Nav1.6 | Nav1.7 | b3 |
| N-[2-(1H- | Tonic | >100 | >100 | >100 | >100 | >100 | >100 | >100 | >100 |
| indol-3-yl)- | Inactivated | 76.0 | 47.6 | 58.7 | >100 | 78.6 | 94.1 | 18.3 | 94.1 |
| ethyl]- | State | | | | | | | | |
| comanilamide | | | | | | | | | |
| N-[2-(5- | Tonic | >100 | >100 | >100 | >100 | >100 | >100 | >100 | >100 |
| methoxy- | Inactivated | | | 71.6 | | | | 87.0 | >100 |
| indol-3-yl)- | State | | | | | | | | |
| ethyl]- | | | | | | | | | |
| comanilamide | | | | | | | | | |
| 2-methyl-4- | Tonic | >100 | >100 | >100 | >100 | >100 | >100 | >100 | >100 |
| oxo-4H-pyran- | Inactivated | | | >100 | | | | 133.2 | >100 |
| 3-yl[2-(5- | State | | | | | | | | |
| methoxy-1H- | | | | | | | | | |
| indol-3- | | | | | | | | | |
| yl)ethyl]carbamate | | | | | | | | | |

### Example 6

Effect of N-[2-(1H-indol-3-yl)-ethyl]-comanilamide, N-[2-(5-methoxy-indol-3-yl)-ethyl]-comanilamide and 2-methyl-4-oxo-4H-pyran-3-yl [2-(5-methoxy-1H-indol-3-yl)ethyl]carbamate on TRPV1 channels.

Method. The ability of N-[2-(1H-indol-3-yl)-ethyl]-comanilamide, N-[2-(5-methoxy-indol-3-yl)-ethyl]-comanilamide and 2-methyl-4-oxo-4H-pyran-3-yl [2-(5-methoxy-1H-indol-3-yl)ethyl]carbamate to act as antagonists of TRPV1 was evaluated with a calcium influx assay. The signal elicited in the presence of the positive control agonist (0.1 μM capsaicin) was set to 100% and the signal in the presence of the antagonist (0.1 μM capsaicin+3 μM ruthenium red) was set to 0. Values were considered significant if the test article mean was three or more standard deviations away from the positive control agonist mean (i.e., greater than 37.74% inhibition).

Results. N-[2-(1H-indol-3-yl)-ethyl]-comanilamide, N-[2-(5-methoxy-indol-3-yl)-ethyl]-comanilamide and 2-methyl-4-oxo-4H-pyran-3-yl [2-(5-methoxy-1H-indol-3-yl)ethyl]carbamate (10 μM) had a significant inhibitory effect (i.e. 37.74% inhibition) on TRPV1 mediated calcium influx (Table 2).

| | Test conc | Normalized | | Normalized | |
|---|---|---|---|---|---|
| Test Article | (μM) | % Activation | SD | % inhibition | SD |
| N-[2-(1H- | 0.1 | 23.07 | 4.66 | 9.88 | 25.02 |
| indo1-3-yl)- | 0.5 | 11.93 | 1.79 | 2.44 | 7.90 |
| ethyl]- | 1 | 10.51 | 6.61 | 31.12 | 1.89 |
| comanilamide | 5 | 8.71 | 6.00 | 26.08 | 10.09 |
| | 10 | 10.13 | 1.05 | 37.74 | 5.57 |

| | Test conc | Normalized | | Normalized | |
|---|---|---|---|---|---|
| Test Article | (μM) | % Activation | SD | % inhibition | SD |
| N-[2-(5- | 0.1 | 18.12 | 4.10 | 15.21 | 17.68 |
| methoxy- | 0.5 | 11.95 | 1.38 | 1.19 | 3.64 |
| indol-3-yl)- | 1 | 14.04 | 4.20 | 36.36 | 4.62 |
| ethyl]- | 5 | 12.92 | 5.98 | 35.39 | 7.47 |
| comanilamide | 10 | 13.67 | 1.52 | 42.59 | 5.39 |

| | Test | | | | |
|---|---|---|---|---|---|
| | conc | Normalized % | | Normalized | |
| Test Article | (μM) | Activation | SD | % inhibition | SD |
| 2-methyl-4-oxo- | 0.1 | 12.65 | 5.35 | −1.78 | 10.57 |
| 4H-pyran-3-yl [2- | 0.5 | 9.58 | 3.56 | 23.56 | 5.32 |
| (5-methoxy-1H- | 1 | 12.16 | 6.56 | 45.10 | 9.63 |
| indol-3- | 5 | 5.75 | 2.03 | 44.60 | 5.13 |
| yl)ethyl]carbamate | 10 | 12.26 | 0.81 | 41.46 | 6.61 |

Conclusions. The compounds of the invention are able to inhibit TRPV1 channels that are involved in itch response.

### Example 7

Preparation of a topical N-[2-(1H-indol-3-yl)-ethyl]-comanilamide cream.

A cream formulation for topical application of N-[2-(1H-indol-3-yl)-ethyl]-comanilamide was prepared by heating 50 g shea butter, 50 g coconut oil in a mason jar until the oils melt and added 3 g (3%) N-[2-(1H-indol-3-yl)-ethyl]-comanilamide until it is dissolved in the oils. The cream was cooled and subsequently whipped on high speed mixer for several minutes for softer texture.

### Example 8

Analgesic/Anti-pruritus Effects in Humans

The anti-pruritus effect of N-[2-(1H-indol-3-yl)-ethyl]-comanilamide was tested using the UVB sunburn model (R. Rolke et al. (2006), Quantitative sensory testing in the German Research Network on neuropathic pain (DFNS): standardized protocol and reference values, Pain, 123, 231-243) used as an art-accepted surrogate model of itch (Andersen et al., Acta Derm Venereol. 2015; 95(7):771-7. Human surrogate models of histaminergic and non-histaminergic itch.).

Twenty four young male subjects (18-N-[2-(1H-indol-3-yl)-ethyl]-comanilamide in escalated doses (300 mg-900 mg) and 6 in the placebo group), were exposed to three times the minimal erythema dose (MED) of UVB light on an approximately 4×4 cm area on the upper part of the leg in the morning of Day (−1) pre-dose on Day 1 and 2.5 hours post—dose on the UVB-irradiated area.

UVB irradiation causes skin inflammation which is noxious and elicits pronounced inflammation related temperature and mechanical hyperalgesia at the UVB-irradiated site. This hyperalgesia peaks at about 20-24 hours post irradiation after which it slowly subsides. Quantitative sensory testing (QST) assesses characteristic sensory patterns in pain and pruritus models.

The UVB irradiated skin was obtained by irradiating the marked area (approximately 4×4 cm) of the skin of the upper part of the right leg by three times the MED as determined during the screening. This was done approximately 20 hours prior to dosing.

Cold pain thresholds (CPT) were determined. The mean threshold temperature of five consecutive measurements was calculated. All thresholds were obtained with ramped stimuli (1 C/s) that were terminated when the subject pressed a button.

Mechanical detection threshold (MDT) to von Frey hairs was assessed by using a standardized set of modified von Frey hairs (Optihair; Marstock Nervtest, Marburg, Germany) exerting forces between 0.25 and 512 mN (rounded tip, 0.5 mm in diameter). Thresholds were determined by the method of limits with the geometric mean of a series of 5 ascending and descending stimulus intensities.

The results were as follows:

Cold pain thresholds levels, pre and post sunburn and 2.5h post N-[2-(1H-indol-3-yl)-ethyl]-comanilamide administration.

| | | | 2.5 h Post |
|---|---|---|---|
| | Pre | Post | Drug |
| CPT (° C.) | sunburn | sunburn | Administration |
| 600 mg | 5.55 | 16.03 | 12.75 |
| Placebo | 13.54 | 16.85 | 15.28 |

Change from baseline of MDT (feel sensation) (mN) post sunburn and 2.5h post N-[2-(1H-indol-3-yl)-ethyl]-comanilamide administration.

MDT feel sensation (mN)

| | 300 mg | 600 mg | 900 mg | Placebo |
|---|---|---|---|---|
| Post sunburn - | −10.92 | −3.52 | −7.24 | −11.57 |
| Change | | | | |
| from | | | | |
| Baseline* | | | | |
| 2.5 h post | −12.13 | −0.017 | 0.84 | −8.57 |
| Drug | | | | |
| administration - | | | | |
| Change | | | | |
| from | | | | |
| Baseline* | | | | |

*Baseline - Day (−1) before the sunburn.

Conclusions. These results clearly demonstrated the analgesic/anti-pruritus effects of N-[2-(1H-indol-3-yl)-ethyl]-comanilamide in human testing, with a peak response at 2.5h post dosing.

Various embodiments of the invention comprise:

1. A method for treating a subject suffering from itch or pruritus, comprising administering to the subject a composition comprising an effective amount of a compound having the formula

Ar—B—Ar′ (I)

wherein:
—B— represents:

### —X—Y—Z—,

wherein:
X represents —(CH₂)ₙ (wherein n is 0-6);
Y represents oxygen, sulphur, >NH or is absent;
Z represents >C═O, >O or >COO or is absent; and wherein at least one of X, Y and
Z must be present;
Ar represents an indole nucleus ring system:

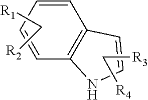

Ar′ represents an alpha-, beta- or gamma-pyrone nucleus ring system:

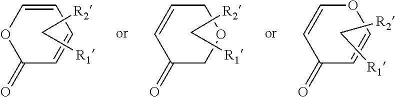

wherein each of R₁₋₄ substitutes the ring system Ar at any available position (including the N-position) and each of R_{1′}R_{2′} substitutes the ring system Ar′ at any available position and wherein each of R₁₋₄ and R_{1′-2′} independently represents hydrogen, oxygen, halo, halo-C₁₋₅ alkyl, aryl, acyl, a C₅₋₇ heterocyclic group containing 1-3 hetero atoms independently selected from nitrogen, oxygen or sulphur; a C₆₋₈ heteroaryl group containing 1-3 hetero atoms independently selected from nitrogen, oxygen or sulphur, C₁₋₅ alkyl, C₂₋₅ alkenyl, C₂₋₅ alkynyl, aryl-C₁₋₅ alkyl, aryl-C₂₋₅ alkenyl, aryl-C₁₋₅ alkynyl, hydroxy-C₁₋₅ alkyl, nitro, amino, cyano, cyanamide, guanidine, amidino, acylamido, C₁₋₅ alkylamine, C₁₋₅ alkylamido, hydroxy, thiol, acyloxy, azido, C₁₋₅ alkoxy, carboxy, carbonylamido or styryl; wherein said arylalkyl, arylalkenyl, aralalkynyl, or styryl group optionally can be ring-substituted by one to four substituents independently selected from the group consisting of hydrogen, halo, halo-C₁₋₅ alkyl, aryl, a C₅₋₇ heterocyclic group containing 1-3 hetero atoms independently selected from nitrogen, oxygen and sulphur; a heteroaryl group containing 1-3 hetero atoms independently selected from nitrogen, oxygen and sulphur; C₁₋₅ alkyl, C₂₋₅ alkenyl, C₂₋₅ alkynyl, aryl-C₁₋₅ alkyl, aryl-C₂₋₅ alkenyl, aryl-C₂₋₅alkynyl, hydroxy-C₁₋₅ alkyl, nitro, amino, cyano, cyanamido, guanidino, amidino, acylamido, hydroxy, thiol, acyloxy, azido, alkoxy, carboxy, carbonylamido, S-alkyl or alkylthiol;
and either of R₃ or R₄ further can include or represent a bond to B;
wherein Ar can be bonded to B at any position on the five-membered ring portion of the Ar ring, including the N-position, and Ar′ can be bonded to B at any carbon on the Ar′ ring not substituted by R_{1′} and R₂′;
or a salt, stereoisomer, or racemic mixture thereof.

2. The method of embodiment 1, wherein X is —(CH₂)ₙ, wherein n is 0-6, Y is >NH or >O and Z is >CO.

3. The method of embodiment 1 or 2, wherein Ar′ is an alpha-pyrone ring system.

4. The method of any of embodiments 1 to 3, wherein Ar′ is a beta-pyrone ring system.

5. The method of any of embodiments 1 to 4, wherein Ar′ is a gamma-pyrone ring system.

6. The method of any of embodiments 1 to 5, wherein X is —(CH₂)ₙ, Y is >NH or >O, and Z is >CO, Ar is an indole ring; R₃ is a bond to X on position 3 of the indole ring; R₁ is hydrogen or a methoxy group on position 5 of the indole ring, and each of R₂ and R₄ is hydrogen; Ar′ is a gamma-pyrone ring bonded to Z at position 2 of the pyrone ring; R₁ is hydrogen or a hydroxy group on position 5 of the pyrone ring; and R₂ is hydrogen or a carboxy group on position 6 of the gamma-pyrone ring; or a pharmaceutically acceptable salt, stereoisomer, or racemic mixture thereof.

7. The method of any of embodiments 1 to 6, wherein X is —(CH₂)ₙ, Y is >NH or >O and Z is >COO; Ar is an indole ring; R₃ is a bond to X on position 3 of the indole ring; R₁ is a methoxy group at position 5 of the indole ring and each of R₂ and R₄ is hydrogen; Ar′ is an gamma-pyrone ring substituted by Z at position 4 of the pyrone ring; and R₁, and R₂ are each a methyl or hydrogen; or a pharmaceutically acceptable salt, stereoisomer, or racemic mixture thereof.

8. The method of any of embodiments 1 to 7, wherein the composition is further characterized by at least one of the following features:

(i) it is adapted for oral, rectal, parenteral, transbuccal, intrapulmonary (e.g. by inhalation) transdermal or topical administration;
(ii) it is in unit dosage form, each unit dosage comprising an effective amount of said compound;
(iii) it is a prolonged release formulation;
(iv) it is in a depot form which will release the compound slowly in the body, over a preselected time period;
(v) it is an ointment, cream, gel, emulsion, oil, foam, solution or aerosol spray suitable for topical use;
(vi) it further comprises at least one additional therapeutic agent selected from a UV protectant, an analgesic, a tranquilizer, a vasoconstrictor, a vasodilator, and an anti-inflammatory agent.

9. The method of any of embodiments 1 to 8, wherein the composition comprises at least one pharmaceutically acceptable diluent, preservative, antioxidant, solubilizer, emulsifier, gelling agent, adjuvant or carrier.

10. A pharmaceutical composition comprising an effective amount of a compound having the formula:

Ar—B—Ar′ (I)

wherein:
—B— represents:

### —X—Y—Z—,

wherein:
X represents —(CH₂)ₙ (wherein n is 0-6);
Y represents oxygen, sulphur, >NH or is absent;
Z represents >C═O, >O or >COO or is absent; and wherein at least one of X, Y and
Z must be present;
Ar represents an indole nucleus ring system:

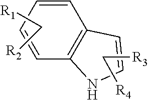

Ar′ represents an alpha-, beta- or gamma-pyrone nucleus ring system:

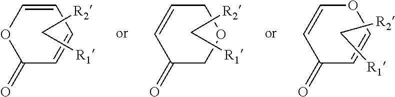

wherein each of R₁₋₄ substitutes the ring system Ar at any available position (including the N-position) and each of R_{1′}-R_{2′} substitutes the ring system Ar′ at any available position and wherein each of R₁₋₄ and R_{1′-2′} independently represents hydrogen, oxygen, halo, halo-C₁₋₅ alkyl, aryl, acyl, a C₅₋₇ heterocyclic group containing 1-3 hetero atoms independently selected from nitrogen, oxygen or sulphur; a C₆₋₈ heteroaryl group containing 1-3 hetero atoms independently selected from nitrogen, oxygen or sulphur, C₁₋₅ alkyl, C₂₋₅ alkenyl, C₂₋₅ alkynyl, aryl-C₁₋₅ alkyl, aryl-C₂₋₅ alkenyl, aryl-C₁₋₅ alkynyl, hydroxy-C₁₋₅ alkyl, nitro, amino, cyano, cyanamide, guanidine, amidino, acylamido, C₁₋₅ alkylamine, C₁₋₅ alkylamido, hydroxy, thiol, acyloxy, azido, C₁₋₅ alkoxy, carboxy, carbonylamido or styryl; wherein said arylalkyl, arylalkenyl, aralalkynyl, or styryl group optionally can be ring-substituted by one to four substituents independently selected from the group consisting of hydrogen, halo, halo-C₁₋₅ alkyl, aryl, a C₅₋₇ heterocyclic group containing 1-3 hetero atoms independently selected from nitrogen, oxygen and sulphur; a heteroaryl group containing 1-3 hetero atoms independently selected from nitrogen, oxygen and sulphur; C₁₋₅ alkyl, C₂₋₅ alkenyl, C₂₋₅ alkynyl, aryl-C₁₋₅ alkyl, aryl-C₂₋₅ alkenyl, aryl-C₂₋₅alkynyl, hydroxy-C₁₋₅ alkyl, nitro, amino, cyano, cyanamido, guanidino, amidino, acylamido, hydroxy, thiol, acyloxy, azido, aikoxy, carboxy, carbonylamido, S-alkyl or alkylthiol;
and either of R₃ or R₄ further can include or represent a bond to B;
wherein Ar can be bonded to B at any position on the five-membered ring portion of the Ar ring, including the N-position, and Ar′ can be bonded to B at any carbon on the Ar′ ring not substituted by R_{1′} and R₂′; or a salt, stereoisomer, or racemic mixture thereof, and at least one pharmaceutically acceptable diluent, preservative, antioxidant, solubilizer, emulsifier, gelling agent, adjuvant or carrier; wherein the pharmaceutical composition:
(i) is adapted for oral, rectal, parenteral, transbuccal, intrapulmonary (e.g. by inhalation) transdermal or topical administration;
(ii) is in unit dosage form, each unit dosage comprising an effective amount of said compound;
(iii) is a prolonged release formulation;
(iv) is in a depot form which will release the compound slowly in the body, over a preselected time period;
(v) is an ointment, cream, gel, foam, emulsion, oil, solution, or spray suitable for topical use;
(vi) further comprises at least one additional therapeutic agent selected from a UV protectant, an analgesic, a tranquilizer, a vasoconstrictor, a vasodilator, and an anti-inflammatory agent; and/or
(vii) is a solid dosage form for oral administration.

11. The pharmaceutical composition of embodiment 10, wherein the composition is an oral dosage form selected from capsules, tablets, pills, powders or granules.

12. The pharmaceutical composition of embodiment 10 or 11, wherein the compound of formula I is admixed with at least one inert pharmaceutically acceptable carrier selected from sucrose, lactose, or starch.

13. The pharmaceutical composition of any of embodiments 10 to 12, wherein the composition further comprises one or more lubricating agents.

14. The pharmaceutical composition of any of embodiments 10 to 13, wherein the composition comprises magnesium stearate.

15. The pharmaceutical composition of any of embodiments 10 to 14, wherein the composition comprises one or more adjuvants.

16. The pharmaceutical composition of any of embodiments 10 to 15, wherein the composition comprises gum tragacanth, acacia, corn starch or gelatin.

17. The pharmaceutical composition of any of embodiments 10 to 16, wherein the composition comprises at least one of an excipient, a disintegrating agent, a lubricant, a sweetening agent, and a flavoring agent.

18. The pharmaceutical composition of any embodiment 17, wherein the excipient is microcrystalline cellulose.

19. The pharmaceutical composition of embodiment 17, wherein the disintegrating agent is corn starch, pregelatinized starch, alginic acid, or a combination thereof.

20. The pharmaceutical composition of embodiment 17, wherein the sweetening agent is sucrose, lactose, saccharin or a combination thereof.

21. The pharmaceutical composition of embodiment 17, wherein the flavoring agent is peppermint, oil of wintergreen, cherry, or a combination thereof.

22. The pharmaceutical composition of any of embodiments 10 to 21, further comprising at least one buffering agent.

23. The pharmaceutical composition of any of embodiments 10 to 22, further comprising a fatty oil.

24. The pharmaceutical composition of any of embodiments 10 to 23, wherein the composition is a topical formulation in the form of an ointment, gel, cream, emulsion, oil, foam or spray for dermal application.

25. The pharmaceutical composition of any of embodiments 10 to 24, wherein the compound is administered in the form of a medicament, which comprises also at least one pharmaceutically acceptable diluent, preservative, antioxidant, solubilizer, emulsifier adjuvant or carrier.

26. The pharmaceutical composition of any of embodiments 10 to 25, wherein the medicament is further characterized by at least one of the following features:

(i) it is adapted for oral, rectal, parenteral, transbuccal, intrapulmonary (e.g. by inhalation) transdermal or ectopic administration;
(ii) it is in unit dosage form, each unit dosage comprising an amount of said at least one compound at effective dose;
(iii) it is a prolonged release formulation;
(iv) it is in a depot form which will release the compound slowly in the body, over a preselected time period;
(v) it is an ointment, cream, foam or spray intended for ectopic use
(vi) it comprises also at least one additional therapeutic agent selected from UV protectants, analgesics, minor tranquilizers, and anti-inflammatory drugs.

27. The method of any of embodiments 1 to 9, wherein the compound is selected from the group consisting of N-[2-(1H-indol-3-yl)-ethyl]-comanilamide, N-[2-(5-methoxy-indol-3-yl)-ethyl]-comanilamide, and 2-methyl-4-oxo-4H-pyran-3-yl [2-(5-methoxy-1H-indol-3-yl)ethyl]carbamate.

28. The method of any of embodiments 1 to 9, wherein itch or pruritus is reduced.

29. The method of any of embodiments 1 to 9, wherein the subject is a human.

30. The method of any of embodiments 1 to 9, wherein the compound inhibits Nav1.7 channels.

31. The method of any of embodiments 1 to 9, wherein the compound inhibits TRPV channels.

32. The pharmaceutical composition of any of embodiments 10 to 26, wherein the compound is selected from the group consisting of N-[2-(1H-indol-3-yl)-ethyl]-comanilamide, N-[2-(5-methoxy-indol-3-yl)-ethyl]-comanilamide, and 2-methyl-4-oxo-4H-pyran-3-yl [2-(5-methoxy-1H-indol-3-yl)ethyl]carbamate.

33. The pharmaceutical composition of any of embodiments 10 to 26, wherein the composition comprises the compound of formula I in an amount sufficient to reduce itch or pruritus in a patient in need of such reduction.

34. The method of any of embodiments 1 to 9, wherein the subject suffers from acute pruritus.

35. The method of any of embodiments 1 to 9, wherein the subject suffers from chronic pruritus.

36. The pharmaceutical composition of any of embodiments 10 to 26, wherein the compound inhibits Nav1.7 channels.

37. The pharmaceutical composition of any of embodiments 10 to 26, wherein the compound inhibits TRPV channels.

38. A cream formulation for topical application comprising shea butter, coconut oil, and a therapeutically effective amount of a pyrone-indole derivative.

39. The cream formulation of embodiment 38, wherein the pyrone-indole derivative is N-[2-(1H-indol-3-yl)-ethyl]-comanilamide.

40. The cream formulation of embodiment 39, wherein the concentration of the N-[2-(1H-indol-3-yl)-ethyl]-comanilamide is 3%.

All citations (e.g., scientific journal publications, patents, and other reference material) mentioned herein are hereby incorporated herein by reference to the same extent as if each individual citation was specifically and individually indicated to be incorporated by reference.

While particular embodiments of the invention have been particularly described hereinabove, it will be appreciated that the present invention is not limited thereto, since as will be readily apparent to skilled persons, many modifications or variations can be made. Such modifications or variations which have not been detailed herein are deemed to be obvious equivalents of the present invention.

### REFERENCES

Akiyama, T., M. I. Carstens and E. Carstens (2010). “Differential itch- and pain-related behavioral responses and micro-opoid modulation in mice.” Acta Derm Venereol 90(6): 575-581.
Bautista, D. M., S. R. Wilson and M. A. Hoon (2014). “Why we scratch an itch: the molecules, cells and circuits of itch.” Nat Neurosci 17(2): 175-182.
Chuquilin, M., Y. Alghalith and K. H. Fernandez (2016). “Neurocutaneous disease: Cutaneous neuroanatomy and mechanisms of itch and pain.” J Am Acad Dermatol 74(2): 197-212.
Furue, M. and T. Kadono (2015). “New therapies for controlling atopic itch.” J Dermatol 42(9): 847-850.
Gibson, R. A., J. Robertson, H. Mistry, S. McCallum, D. Fernando, M. Wyres and G. Yosipovitch (2014). “A randomised trial evaluating the effects of the TRPV1 antagonist SB705498 on pruritus induced by histamine, and cowhage challenge in healthy volunteers.” PLoS One 9(7): e100610.
Lauffer, F. and J. Ring (2016). “Target-oriented therapy: Emerging drugs for atopic dermatitis.” Expert Opin Emerg Drugs 21(1): 81-89.
Lavery, M. J., C. Stull, M. O. Kinney and G. Yosipovitch (2016). “Nocturnal Pruritus: The Battle for a Peaceful Night's Sleep.” Int J Mol Sci 17(3).
Lee, J. H., C. K. Park, G. Chen, Q. Han, R. G. Xie, T. Liu, R. R. Ji and S. Y. Lee (2014). “A monoclonal antibody that targets a NaV1.7 channel voltage sensor for pain and itch relief.” Cell 157(6): 1393-1404.
Maurer, M., K. Rosen, H. J. Hsieh, S. Saini, C. Grattan, A. Gimenez-Arnau, S. Agarwal, R. Doyle, J. Canvin, A. Kaplan and T. Casale (2013). “Omalizumab for the treatment of chronic idiopathic or spontaneous urticaria.” N Engl J Med 368(10): 924-935.
Rice, F. L., P. J. Albrecht, J. P. Wymer, J. A. Black, I. S. Merkies, C. G. Faber and S. G. Waxman (2015). “Sodium channel Nav1.7 in vascular myocytes, endothelium, and innervating axons in human skin.” Mol Pain 11: 26.

1. A method for treating a subject suffering from itch or pruritus, comprising administering to the subject a composition comprising an effective amount of a compound having the formula
Ar—B—Ar′ (I)
wherein:
—B— represents:
—X—Y—Z—,
wherein:
X represents —(CH₂)ₙ (wherein n is 0-6);
Y represents oxygen, sulphur, >NH or is absent;
Z represents >C═O, >O or >COO or is absent; and wherein at least one of X, Y and Z must be present;
Ar represents an indole nucleus ring system: 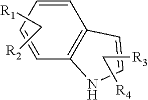
Ar′ represents an alpha-, beta- or gamma-pyrone nucleus ring system: 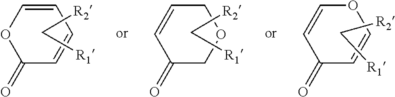
wherein each of R₁₋₄ substitutes the ring system Ar at any available position (including the N-position) and each of R_{1′}-R_{2′} substitutes the ring system Ar′ at any available position and wherein each of R₁₋₄ and R_{1′-2′} independently represents hydrogen, oxygen, halo, halo-C₁₋₅ alkyl, aryl, acyl, a C₅₋₇ heterocyclic group containing 1-3 hetero atoms independently selected from nitrogen, oxygen or sulphur; a C₆₋₈ heteroaryl group containing 1-3 hetero atoms independently selected from nitrogen, oxygen or sulphur, C₁₋₅ alkyl, C₂₋₅ alkenyl, C₂₋₅ alkynyl, aryl-C₁₋₅ alkyl, aryl-C₂₋₅ alkenyl, aryl-C₁₋₅alkynyl, hydroxy-C₁₋₅ alkyl, nitro, amino, cyano, cyanamide, guanidine, amidino, acylamido, C₁₋₅ alkylamine, C₁₋₅ alkylamido, hydroxy, thiol, acyloxy, azido, C₁₋₅alkoxy, carboxy, carbonylamido or styryl; wherein said arylalkyl, arylalkenyl, aralalkynyl, or styryl group optionally can be ring-substituted by one to four substituents independently selected from the group consisting of hydrogen, halo, halo-C₁₋₅ alkyl, aryl, a C₅₋₇ heterocyclic group containing 1-3 hetero atoms independently selected from nitrogen, oxygen and sulphur; a heteroaryl group containing 1-3 hetero atoms independently selected from nitrogen, oxygen and sulphur; C₁₋₅ alkyl, C₂₋₅ alkenyl, C₂₋₅ alkynyl, aryl-C₁₋₅ alkyl, aryl-C₂₋₅ alkenyl, aryl-C₂₋₅alkynyl, hydroxy-C₁₋₅alkyl, nitro, amino, cyano, cyanamido, guanidino, amidino, acylamido, hydroxy, thiol, acyloxy, azido, aikoxy, carboxy, carbonylamido, S-alkyl or alkylthiol;
and either of R₃ or R₄ further can include or represent a bond to B;
wherein Ar can be bonded to B at any position on the five-membered ring portion of the Ar ring, including the N-position, and Ar′ can be bonded to B at any carbon on the Ar′ ring not substituted by R_{1′} and R₂′;
or a salt, stereoisomer, or racemic mixture thereof.

2. The method of claim 1, wherein X is —(CH₂)ₙ, wherein n is 0-6, Y is >NH or >O and Z is >CO.

3. The method of claim 2, wherein Ar′ is an alpha-pyrone ring system.

4. The method of claim 2, wherein Ar′ is a beta-pyrone ring system.

5. The method of claim 2, wherein Ar′ is a gamma-pyrone ring system.

6. The method of claim 1, wherein X is —(CH₂)ₙ, Y is >NH or >O, and Z is >CO, Ar is an indole ring; R₃ is a bond to X on position 3 of the indole ring; R₁ is hydrogen or a methoxy group on position 5 of the indole ring, and each of R₂ and R₄ is hydrogen; Ar′ is a gamma-pyrone ring bonded to Z at position 2 of the pyrone ring; R₁ is hydrogen or a hydroxy group on position 5 of the pyrone ring; and R₂ is hydrogen or a carboxy group on position 6 of the gamma-pyrone ring; or a pharmaceutically acceptable salt, stereoisomer, or racemic mixture thereof.

7. The method of claim 1, wherein X is —(CH₂)ₙ, Y is >NH or >O and Z is >COO; Ar is an indole ring; R₃ is a bond to X on position 3 of the indole ring; R₁ is a methoxy group at position 5 of the indole ring and each of R₂ and R₄ is hydrogen; Ar′ is an gamma-pyrone ring substituted by Z at position 4 of the pyrone ring; and R₁, and R₂ are each a methyl or hydrogen; or a pharmaceutically acceptable salt, stereoisomer, or racemic mixture thereof.

8. The method of claim 1, wherein the composition is further characterized by at least one of the following features:
(i) it is adapted for oral, rectal, parenteral, transbuccal, intrapulmonary (e.g. by inhalation) transdermal or topical administration;
(ii) it is in unit dosage form, each unit dosage comprising an effective amount of said compound;
(iii) it is a prolonged release formulation;
(iv) it is in a depot form which will release the compound slowly in the body, over a preselected time period;
(v) it is an ointment, cream, gel, emulsion, oil, foam, solution or aerosol spray suitable for topical use;
(vi) it further comprises at least one additional therapeutic agent selected from a UV protectant, an analgesic, a tranquilizer, a vasoconstrictor, a vasodilator, and an anti-inflammatory agent.

9. The method of claim 1, wherein the composition comprises at least one pharmaceutically acceptable diluent, preservative, antioxidant, solubilizer, emulsifier, gelling agent, adjuvant or carrier.

10. A pharmaceutical composition comprising an effective amount of a compound having the formula:
Ar—B—Ar′ (I)
wherein:
—B— represents:
—X—Y—Z—,
wherein:
X represents —(CH₂)ₙ (wherein n is 0-6);
Y represents oxygen, sulphur, >NH or is absent;
Z represents >C═O, >O or >COO or is absent; and wherein at least one of X, Y and Z must be present;
Ar represents an indole nucleus ring system: 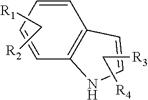
Ar′ represents an alpha-, beta- or gamma-pyrone nucleus ring system: 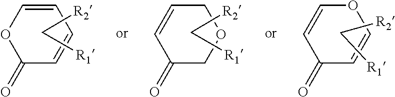
wherein each of R₁₋₄ substitutes the ring system Ar at any available position (including the N-position) and each of R_{1′}-R_{2′} substitutes the ring system Ar′ at any available position and wherein each of R₁₋₄ and R_{1′-2′} independently represents hydrogen, oxygen, halo, halo-C₁₋₅ alkyl, aryl, acyl, a C₅₋₇ heterocyclic group containing 1-3 hetero atoms independently selected from nitrogen, oxygen or sulphur; a C₆₋₈ heteroaryl group containing 1-3 hetero atoms independently selected from nitrogen, oxygen or sulphur, C₁₋₅ alkyl, C₂₋₅ alkenyl, C₂₋₅ alkynyl, aryl-C₁₋₅ alkyl, aryl-C₂₋₅ alkenyl, aryl-C₁₋₅alkynyl, hydroxy-C₁₋₅ alkyl, nitro, amino, cyano, cyanamide, guanidine, amidino, acylamido, C₁₋₅ alkylamine, C₁₋₅ alkylamido, hydroxy, thiol, acyloxy, azido, C₁₋₅alkoxy, carboxy, carbonylamido or styryl; wherein said arylalkyl, arylalkenyl, aralalkynyl, or styryl group optionally can be ring-substituted by one to four substituents independently selected from the group consisting of hydrogen, halo, halo-C₁₋₅ alkyl, aryl, a C₅₋₇ heterocyclic group containing 1-3 hetero atoms independently selected from nitrogen, oxygen and sulphur; a heteroaryl group containing 1-3 hetero atoms independently selected from nitrogen, oxygen and sulphur; C₁₋₅ alkyl, C₂₋₅ alkenyl, C₂₋₅ alkynyl, aryl-C₁₋₅alkyl, aryl-C₂₋₅ alkenyl, aryl-C₂₋₅alkynyl, hydroxy-C₁₋₅alkyl, nitro, amino, cyano, cyanamido, guanidino, amidino, acylamido, hydroxy, thiol, acyloxy, azido, aikoxy, carboxy, carbonylamido, S-alkyl or alkylthiol;
and either of R₃ or R₄ further can include or represent a bond to B;
wherein Ar can be bonded to B at any position on the five-membered ring portion of the Ar ring, including the N-position, and Ar′ can be bonded to B at any carbon on the Ar′ ring not substituted by R_{1′} and R_{2′}; or a salt, stereoisomer, or racemic mixture thereof, and at least one pharmaceutically acceptable diluent, preservative, antioxidant, solubilizer, emulsifier, gelling agent, adjuvant or carrier;
wherein the pharmaceutical composition:
(i) is adapted for oral, rectal, parenteral, transbuccal, intrapulmonary (e.g. by inhalation) transdermal or topical administration;
(ii) is in unit dosage form, each unit dosage comprising an effective amount of said compound;
(iii) is a prolonged release formulation;
(iv) is in a depot form which will release the compound slowly in the body, over a preselected time period;
(v) is an ointment, cream, gel, foam, emulsion, oil, solution, or spray suitable for topical use;
(vi) further comprises at least one additional therapeutic agent selected from a UV protectant, an analgesic, a tranquilizer, a vasoconstrictor, a vasodilator, and an anti-inflammatory agent; and/or
(vii) is a solid dosage form for oral administration.

11. The pharmaceutical composition of claim 10, wherein the composition is an oral dosage form selected from capsules, tablets, pills, powders or granules.

12. The pharmaceutical composition of claim 11, wherein the compound of formula I is admixed with at least one inert pharmaceutically acceptable carrier selected from sucrose, lactose, or starch.

13. The pharmaceutical composition of claim 12, wherein the composition further comprises one or more lubricating agents.

14. The pharmaceutical composition of claim 11, wherein the composition comprises magnesium stearate.

15. The pharmaceutical composition of claim 11, wherein the composition comprises one or more adjuvants.

16. The pharmaceutical composition of claim 11, wherein the composition comprises gum tragacanth, acacia, corn starch or gelatin.

17. The pharmaceutical composition of claim 11, wherein the composition comprises at least one of an excipient, a disintegrating agent, a lubricant, a sweetening agent, and a flavoring agent.

18. The pharmaceutical composition of claim 17, wherein the excipient is microcrystalline cellulose.

19. The pharmaceutical composition of claim 17, wherein the disintegrating agent is corn starch, pregelatinized starch, alginic acid, or a combination thereof.

20. The pharmaceutical composition of claim 17, wherein the sweetening agent is sucrose, lactose, saccharin or a combination thereof.

21. The pharmaceutical composition of claim 17, wherein the flavoring agent is peppermint, oil of wintergreen, cherry, or a combination thereof.

22. The pharmaceutical composition of claim 11, further comprising at least one buffering agent.

23. The pharmaceutical composition of claim 11, further comprising a fatty oil.

24. The pharmaceutical composition of claim 10, wherein the composition is a topical formulation in the form of an ointment, gel, cream, emulsion, oil, foam or spray for dermal application.

25. The pharmaceutical composition of claim 24, wherein the compound is administered in the form of a medicament, which comprises also at least one pharmaceutically acceptable diluent, preservative, antioxidant, solubilizer, emulsifier adjuvant or carrier.

26. The pharmaceutical composition of claim 25, wherein the medicament is further characterized by at least one of the following features:
(i) it is adapted for oral, rectal, parenteral, transbuccal, intrapulmonary (e.g. by inhalation) transdermal or ectopic administration;
(ii) it is in unit dosage form, each unit dosage comprising an amount of said at least one compound at effective dose;
(iii) it is a prolonged release formulation;
(iv) it is in a depot form which will release the compound slowly in the body, over a preselected time period;
(v) it is an ointment, cream, foam or spray intended for ectopic use;
(vi) it comprises also at least one additional therapeutic agent selected from UV protectants, analgesics, minor tranquilizers, and anti-inflammatory drugs.

27. The method of claim 1, wherein the compound is selected from the group consisting of N-[2-(1H-indol-3-yl)-ethyl]-comanilamide, N-[2-(5-methoxy-indol-3-yl)-ethyl]-comanilamide, and 2-methyl-4-oxo-4H-pyran-3-yl [2-(5-methoxy-1H-indol-3-yl)ethyl]carbamate.

28-31. (canceled)

32. The pharmaceutical composition of claim 10, wherein the compound is selected from the group consisting of N-[2-(1H-indol-3-yl)-ethyl]-comanilamide, N-[2-(5-methoxy-indol-3-yl)-ethyl]-comanilamide, and 2-methyl-4-oxo-4H-pyran-3-yl [2-(5-methoxy-1H-indol-3-yl)ethyl]carbamate.

33. The pharmaceutical composition of claim 10, wherein the composition comprises the compound of formula I in an amount sufficient to reduce itch or pruritus in a patient in need of such reduction.

34-37. (canceled)

38. A cream formulation for topical application comprising shea butter, coconut oil, and a therapeutically effective amount of a pyrone-indole derivative.

39. The cream formulation of claim 38, wherein the pyrone-indole derivative is N-[2-(1H-indol-3-yl)-ethyl]-comanilamide.

40. The cream formulation of claim 39, wherein the concentration of the N-[2-(1H-indol-3-yl)-ethyl]-comanilamide is 3%.
